(19) United States
(12) Patent Application Publication
(10) Pub. No.: US 2021/0106704 A1
(43) Pub. Date: Apr. 15, 2021

(54) SYSTEM TO STERILIZE STETHOSCOPES

(71) Applicants: Clifton Schermerhorn, San Juan Capistrano, CA (US); Brock Schermerhorn, San Juan Capistrano, CA (US); Barry Lyou, San Juan Capistrano, CA (US)

(72) Inventors: Clifton Schermerhorn, San Juan Capistrano, CA (US); Brock Schermerhorn, San Juan Capistrano, CA (US); Barry Lyou, San Juan Capistrano, CA (US)

(21) Appl. No.: 16/653,954

(22) Filed: Oct. 15, 2019

(51) Int. Cl. A61L 2/10; A61L 2/26; A61L 2/20

(52) U.S. Cl. CPC A61L 2/10; A61L 2/26; A61L 2/202; A61L 2202/24; A61L 2202/122; A61L 2202/11; A61L 2202/121

(57) ABSTRACT

An illustrated view of an exemplary sanitizing system for sanitizing medical equipment is presented. The sanitizing system is useful for sanitizing medical equipment, such as stethoscopes, of harmful and often times life-threatening pathogens such as MRSA and C-DIFF. The sanitizing system provides a chemical free sanitization while being easy to use and efficient. The sanitizing system produces a quick and easy way to sanitize medical equipment and thereby prevent infectious contaminants to both medical personnel and patients. The sanitizing system can be used in hospital settings, doctor's offices, clinics, ambulances, etc.

### FIELD OF THE INVENTION

This invention relates to stethoscopes. More particularly, it relates to a system to sterilize stethoscopes.

### BACKGROUND

The stethoscope is an acoustic medical device for auscultation, or listening to the internal sounds of an animal or human body. It typically has a small disc-shaped resonator that is placed against the skin, and one or two tubes connected to two earpieces. A stethoscope can be used to listen to the sounds made by the heart, lungs or intestines, as well as blood flow in arteries and veins. In combination with a manual sphygmomanometer, it is commonly used when measuring blood pressure.

Less commonly, “mechanic's stethoscopes”, equipped with rod shaped chest-pieces, are used to listen to internal sounds made by machines (for example, sounds and vibrations emitted by worn ball bearings), such as diagnosing a malfunctioning automobile engine by listening to the sounds of its internal parts. Stethoscopes can also be used to check scientific vacuum chambers for leaks and for various other small-scale acoustic monitoring tasks.

A stethoscope that intensifies auscultatory sounds is called a phonendoscope.

With the rise of Methicillin-Resistant Staphylococcus Aureus (MRSA), Clostridium Difficile (C-DIFF) as well as other pathogens has presented a challenge for the healthcare industry. These pathogens are becoming increasingly common in healthcare environments.

In addition to being difficult to treat with traditional antibiotics, the later has even become resistant to the alcohol-based hand sanitizers used as an alternative to hand washing. In 2011, there were almost a half million C-DIFF infections in the United States. Of these, 83,000 had at least one recurrence and 29,000 died within 30 days of diagnosis. With respect to MRSA, it is estimated that 2 percent of the population are carriers of this pathogen. Although the annual number of cases has decreased, it still remains a significant threat, capable of causing extremely fast developing and often lethal bloodstream infections, pneumonia and surgical site infections.

In light of the forgoing, there is a need for a system that can efficiently and easily sanitize hospital equipment and other medical equipment, such as stethoscopes, that can be subject to dangerous pathogens such as MRSA and C-DIFF.

### BRIEF DESCRIPTION OF THE DRAWINGS

FIG. 1A an illustrated view of an exemplary sanitizing system.

FIG. 1B is an illustrated view of a first door and second door of the sanitizing system shown in FIG. 1A.

The phrases “in one embodiment,” “in various embodiments,” “in some embodiments,” and the like are used repeatedly. Such phrases do not necessarily refer to the same embodiment. The terms “comprising,” “having,” and “including” are synonymous, unless the context dictates otherwise. Such terms do not generally signify a closed list.

“Above,” “adhesive,” “affixing,” “any,” “around,” “both,” “bottom,” “by,” “comprising,” “consistent,” “customized,” “enclosing,” “friction,” “in,” “labeled,” “lower,” “magnetic,” “marked,” “new,” “nominal,” “not,” “of,” “other,” “outside,” “outwardly,” “particular,” “permanently,” “preventing,” “raised,” “respectively,” “reversibly,” “round,” “square,” “substantial,” “supporting,” “surrounded,” “surrounding,” “threaded,” “to,” “top,” “using,” “wherein,” “with,” or other such descriptors herein are used in their normal yes-or-no sense, not as terms of degree, unless context dictates otherwise.

Reference is now made in detail to the description of the embodiments as illustrated in the drawings. While embodiments are described in connection with the drawings and related descriptions, there is no intent to limit the scope to the embodiments disclosed herein. On the contrary, the intent is to cover all alternatives, modifications and equivalents. In alternate embodiments, additional devices, or combinations of illustrated devices, may be added to, or combined, without limiting the scope to the embodiments disclosed herein.

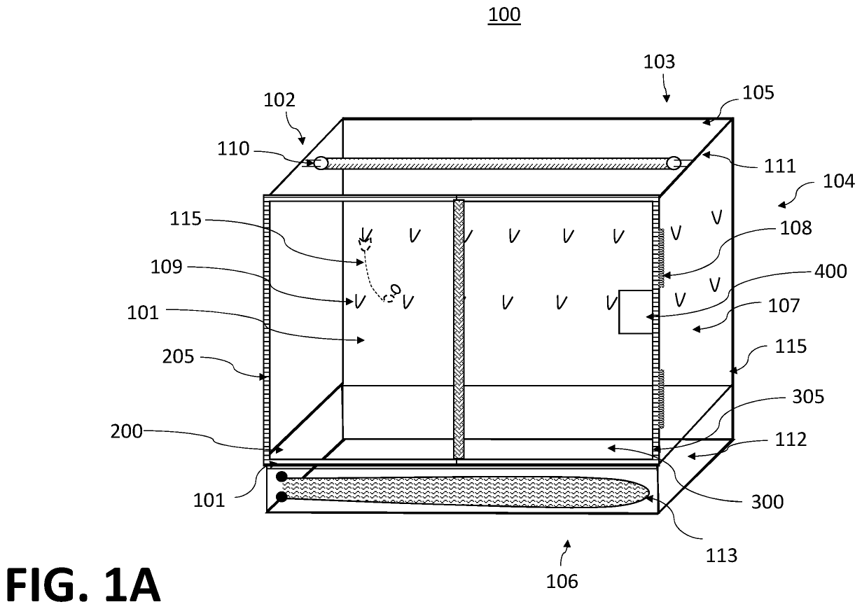

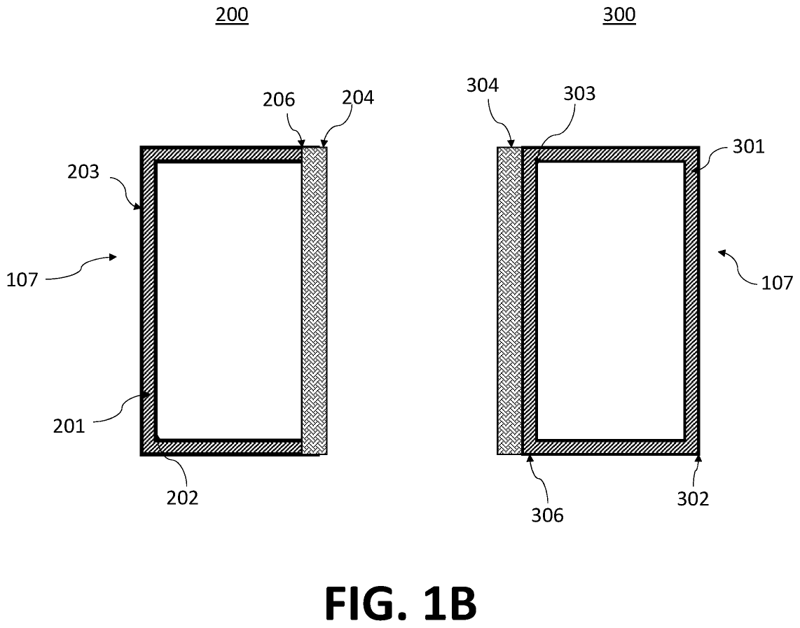

Referring to FIG. 1A and FIG. 1B, an illustrated view of an exemplary sanitizing system 100 for sanitizing medical equipment is presented. The sanitizing system 100 is useful for sanitizing medical equipment, such as stethoscopes, of harmful and often times life-threatening pathogens such as MRSA and C-DIFF. The sanitizing system 100 provides a chemical free sanitization while being easy to use and efficient. The sanitizing system 100 produces a quick and easy way to sanitize medical equipment and thereby prevent infectious contaminants to both medical personnel and patients. The sanitizing system can be used in hospital settings, doctor's offices, clinics, ambulances, etc.

The sanitizing system 100 preferably is made of plastic polymers and metal, however other materials are hereby contemplated, including, but not limited to, medical grade high-density polyethylene (HDPE), low-density polyethylene (LDPE), etc. The sanitizing system 100 preferably has a height of twenty-four (24) inches, however other heights are hereby contemplated, including, but not limited to, twenty (20) inches, twenty-seven (27) inches, etc. The sanitizing system 100 preferably has a length of thirty (30) inches, however other lengths are hereby contemplated, including, but not limited to, twenty-eight (28) inches, thirty-three (33) inches, etc. The sanitizing system 100 preferably has a width of six (6) inches, however other widths are hereby contemplated, including, but not limited to, five (5) inches, eight (8) inches, etc. The sanitizing system 100 preferably has a rectangular shape, however other shapes are hereby contemplated, including, but not limited to, square, trapezoidal, etc.

The sanitizing system 100 has a plurality of sides 101, 102, 103, 104, a top 105, a bottom 106, an inside 107 and a plurality of hinges 108.

A first of the sides 101 has a first door 200 and a second door 300. The first door 200 and the second door 300 are configured to allow access to inside 107 of the sanitizing system 100. A second end 205 of the first door 200 is coupled to the second of the sides 102 by the hinges 108. A second end 305 of the second door 300 is coupled to the fourth of the sides 104 by the hinges 108. The hinges 108 are configured to open and close the first door 200 and the second door 300 without the first door 200 and the second door 300 from becoming disengaged with the sanitizing system 100.

In FIG. 1B, an illustrated view of the first door 200 and the second door 300 of the sanitizing system 100 shown in FIG. 1A is presented.

The first door 200 has a frame 201. The frame 201 has an exterior side 202 and an interior side 203. The exterior side 202 of the first door 200 is configured to be exposed. The exterior side 202 of the first door 200 is configured to be facing the inside 107 of the sanitizing system 100. The exterior side 202 has an extension 204 on a first side 206 of the first door 200.

The second door 300 has a frame 301. The frame 301 has an exterior side 302 and an interior side 303. The exterior side 302 of the second door 300 is configured to be exposed. The interior side 303 of the second door 300 is configured to be facing the inside 107 of the sanitizing system 100. The interior side 303 has an extension 304 on a first side 306 of the second door 300. When the first door 200 and the second door 300 are closed, the extension 204 of the first door 200 is securely coupled to the extension 304 of the second door 300 thereby providing a barrier for air to enter or exit from the sanitizing system 100.

Now referring to FIG. 1A, the inside 107 of the sanitizing system 100 has a plurality of hooks 109, a ultraviolet light 110, a top 111, a bottom 112 and an ozone generator 113.

The plurality of hooks 109 are coupled to a back 114 of the inside 107 of the sanitizing system 100. The hooks 109 are configured to hold medical devices, such as a stethoscope, while the medical devices are being sanitized.

The ultraviolet light 110 is coupled to the top 111 of the inside 107 of the sanitizing system 100. The ultraviolet light 110 is configured to provide ultraviolet lighting that will sanitize the medical devices from any pathogens which may be coupled to the medical devices, ultraviolet lighting is an effective way to kill pathogens. The ultraviolet light 100 is electrically coupled to a power source (not shown) by an electrical cord 115. The electrical cord 115 is coupled via the third of the sides 103 of the sanitizing system 100.

The ozone generator 113 is coupled to the bottom 112 of the inside 107 of the sanitizing system 100. The ozone generator 113 is configured to expose any pathogens coupled to the medical devices coupled to the hooks 108 to ozone, the ozone is another effective sanitizer for killing pathogens.

A handle 400 is coupled to the second door 300. The handle 400 is configured to be useful for providing portability of the sanitizing system 100. The fourth side 104 of the sanitizing system 100 being coupled to a wall (not shown) by one or more couplings (not shown). Alternatively, the bottom 106 being coupled to a table.

In the numbered clauses below, specific combinations of aspects and embodiments are articulated in a shorthand form such that (1) according to respective embodiments, for each instance in which a “component” or other such identifiers appear to be introduced (with “a” or “an,” e.g.) more than once in a given chain of clauses, such designations may either identify the same entity or distinct entities; and (2) what might be called “dependent” clauses below may or may not incorporate, in respective embodiments, the features of “independent” clauses to which they refer or other features described above.

Those skilled in the art will appreciate that the foregoing specific exemplary processes and/or devices and/or technologies are representative of more general processes and/or devices and/or technologies taught elsewhere herein, such as in the claims filed herewith and/or elsewhere in the present application.

The features described with respect to one embodiment may be applied to other embodiments or combined with or interchanged with the features of other embodiments, as appropriate, without departing from the scope of the present invention.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A sanitizing system for sanitizing medical equipment, the system comprising:
a first side, the first side have a first door and a second door;
a plurality of hinges, wherein a second side of the first door being movably coupled to a second side of the system and a second side of the second door to a fourth side of the system by the plurality of hinges;
an inside, the inside having a back, a top and a bottom;
an ultraviolet lamp, the ultraviolet lamp being coupled to the top of the inside, wherein the ultraviolet lamp being electrically coupled to a power source by an electrical cord;
an ozone generator, the ozone generator being coupled to the bottom of the inside of the system;
a plurality of hooks, the plurality of hooks being coupled to the back of the inside of the system; and
a handle, the handle coupled to the second door.

2. The system of claim 1, wherein the system having a height being twenty-four (24) inches.

3. The system of claim 1, wherein the system having a length being thirty (30) inches.

4. The system of claim 1, wherein the system having a width being six (6) inches.

5. The system of claim 1, wherein the system having a shape being rectangular.

6. The system of claim 1, wherein the system being made of plastic polymers material and metal materials.

7. The system of claim 1, wherein the first door further comprising:
a front side, the front side having an extension; and
a interior side.

8. The system of claim 1, wherein the second door further comprising:
a front side; and
a back side, wherein the back side having an extension.

9. The system of claim 1, wherein the back of the system having a coupling, wherein the system being coupled to a wall.

10. The system of claim 1, wherein the bottom of the system being coupled to a table.
